(19) United States
(12) Patent Application Publication
(10) Pub. No.: US 2026/0200978 A1
(43) Pub. Date: Jul. 16, 2026

(54) PEPTIDE INHIBITORS OF BETA-LACTAMASE

(71) Applicant: The Curators of the University of Missouri, Columbia, MO (US)

(72) Inventors: Hongmin SUN, Columbia, MO (US); Xiaoqin ZOU, Columbia, MO (US); Xiao HENG, Columbia, MO (US); Xianjin XU, Columbia, MO (US)

(21) Appl. No.: 19/135,983

(22) PCT Filed: Nov. 28, 2023
(86) PCT No.: PCT/US23/81327

§ 371 Date: Jun. 5, 2025

Related U.S. Application Data
(60) Provisional application No. 63/386,583, filed on Dec. 8, 2022.

(51) Int. Cl. C07K 7/06; A61K 38/00; A61K 45/06; A61P 31/04; C07K 14/00; C12N 9/99

(52) U.S. Cl. CPC C07K 7/06; A61K 45/06; A61P 31/04; C07K 14/001; C12N 9/99; A61K 38/00; C07K 2319/10

(57) ABSTRACT

Disclosed herein are new peptide inhibitors of beta-lactamase that can improve the efficacy of currently available antibiotics. Methods of using the peptide inhibitors for treating bacterial infections are also disclosed.

### CROSS-REFERENCE TO RELATED APPLICATIONS

This application claims priority under 35 U.S.C. § 119(e) to United States Provisional Patent Application Ser. No. 63/386,583, filed on Dec. 8, 2022, the disclosure of which is hereby incorporated by reference in its entirety.

### STATEMENT REGARDING FEDERALLY SPONSORED RESEARCH OR DEVELOPMENT

This invention was made with Government support under grant numbers U01HL152410 awarded by the National Institutes of Health. The government has certain rights in the invention.

### INCORPORATION OF SEQUENCE LISTING

A computer readable form of the Sequence Listing containing the file named “22UMC076 Sequence Listing.xml”, which is 71,180 bytes in size (as measured in MICROSOFT WINDOWS® EXPLORER), is provided herein and is herein incorporated by reference. This Sequence Listing consists of SEQ ID NOs:1-62.

### BACKGROUND OF THE INVENTION

The present disclosure relates to peptide inhibitors of beta-lactamase that can improve the efficacy of antibiotics and methods of treating bacterial infections using the peptide inhibitors.

The emergence of antibiotic resistance poses an urgent medical problem worldwide. The U.S. Centers for Disease Control and Prevention (CDC) estimates that more than 2.8 million infections and 35,000 deaths each year in the United States. The Organization for Economic Co-operation and Development (OECD) predicted that 2.4 million deaths in Europe, North America and Australia from infections by resistant microorganisms in the next 30 years will cost up to US$3.5 billion per year. β-lactam antibiotics were the first class of natural antibiotics to be developed and remain a major class of drugs in clinical use. However, resistance has developed against β-lactam and production of β-lactamase is the most prominent mechanism of resistance among certain bacteria.

Selective β-lactamase inhibitors have been developed and co-administered with antibiotics to overcome resistance. Augmentin (amoxicillin/clavulanate) has achieved huge commercial success. In 2017, Augmentin (amoxicillin/clavulanate) had more about 6.4 million US prescriptions (https://clincalc.com/DrugStats/) and GSK alone achieved £587 million sale. However, resistance to the inhibitors have been developing in multi-drug resistance (MDR) bacteria. For example, extended-spectrum beta-lactamase (ESBL)-producing Enterobacteriaceae is categorized as a serious threat by CDC, with 197,400 cases identified in US hospitalized patients in 2017. There is tremendous interest and effort in developing β-lactamase inhibitors due to clinical needs. Most of the effort focus on small-molecule chemical compounds. There are few studies on developing other biological agents such as peptides as inhibitors, and the reported peptides have very low inhibitory activity in vitro and no reported inhibitory activity in vivo. Peptide drugs constitute an important source for drug development that cannot be substituted by small molecules, such as in cases that involve a very large or quite flat targeting pocket. Advances of synthetic strategies also significantly improve the potential of peptide drugs by extending half-life or improving solubility.

### BRIEF DESCRIPTION

Provided herein are antibacterial peptides having a binding motif comprising an amino acid sequence selected from the group consisting of: SEQ ID NO:1. SEQ ID NO:2, and combinations thereof.

Also provided are compositions comprising an antibacterial peptide described herein and a pharmaceutically acceptable carrier.

Also provided are methods of reducing a bacterial titer, the methods comprising administering the antibacterial peptide to the bacteria.

Other objects and features will be in part apparent and in part pointed out hereinafter.

### BRIEF DESCRIPTION OF THE DRAWINGS

FIG. 1 is a graph showing the stability of BP100-T61-25 and RI-D-BP100-T61-25 measured in 25% human serum for 0 hours and 24 hours.

FIG. 2 is a graph showing Peptide RI-D-BP100-T61-25 at different concentrations (0 μM to 10 μM) in combination with amoxicillin at different concentrations (0 μg/ml to 32 μg/ml) to inhibit ATCC35218 growth. *P<0.05, **P<0.01.

FIGS. 3A and 3B are graphs showing RI-D-BP100-T61-25 combined with amoxicillin and tested against both SAP231 (FIG. 3A) and NRS384 strain (FIG. 3B).

FIGS. 4A and 4B are graphs showing RI-D-BP100-T61-25 combined with cefazolin and tested against both SAP231 (FIG. 4A) and NRS384 strain (FIG. 4B).

Provided herein are peptides that can inhibit the action of beta lactamase. Certain aspects of the disclosure include novel peptide inhibitors against TEM-1 β-lactamase in E. coli, which will work through a different mechanism from the known small-molecule inhibitors. The class A TEM-1 lactamase is the most prevalent plasmid encoded lactamase in gram-negative bacteria. As a result, these novel peptide inhibitors could potentially replace or supplement the current β-lactamase inhibitor drugs to overcome the MDR bacterial resistance to these drugs, which will meet a significant clinical need.

Certain preferred methods and materials are described below, although methods and material similar or equivalent to those described herein can be used in practice or testing of the present invention. All publications, patent applications, patents and other references mentioned herein are incorporated by reference in their entirety. The materials, methods and examples disclosed herein are illustrative only and not intended to be limiting.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

In various embodiments, an antibacterial peptide is provided. The antibacterial peptide includes an amino acid sequence selected from the group consisting of: dGdTdAdAdAdQdAdLdYdTdK (referred to herein as “all-d(GTAAAQALYTK)” and “RI-D-T61-25” (SEQ ID NO:1), dGdTdAdAdAdQdAdLdYdTdKdLdYdKdLdIdKdKdFdLdKdK (referred to herein as “all-d(GTAAAQALYTKLYKLIKKFLKK)” and “RI-D-BP100-T61-25”) (SEQ ID NO:2), and combinations thereof.

In various embodiments, the antibiotic peptide can further include a cell permeating peptide (CPP). The cell permeating peptide can assist in facilitating the entry of the antibiotic peptide into the target cell (i.e., bacterium). Various cell permeating peptides are known in the art. For example, additional CPPs known in the art can be found on online databases (i.e., crdd.osdd.net/raghava/cppsite), in Oikawa et al., (Screening of a Cell-Penetrating Peptide Library in Escherichia coli: Relationship between Cell Penetration Efficiency and Cytotoxicity. ACS Omega 2018, 3, 16489-164), the disclosure of each is incorporated by reference in its entirety. In various embodiments, the cell permeating peptide can include any peptide listed in Table 2 below. In various embodiments, the cell permeating peptide can include or consist of any one of SEQ ID NOs: 3 to 58. Surprisingly and unexpectedly, reversing the CPP peptide (a “retro inverso cell permeating peptide”) retained the cell permeating activity. Thus, in various embodiments, the cell permeating peptide can include the reverse of any peptide listed in Table 2 below. As used herein, the term cell permeating peptide includes forward cell permeating peptides and retro inverso cell permeating peptides. For example, the CFFKDEL (SEQ ID NO:4) cell permeating peptide is an example of a forward cell permeating peptide and LEDKFFC (SEQ ID NO:58) cell permeating peptide is an example of its retro inverso (RI) sequence. Additionally. “cell permeating peptide” is also intended to include cell permeating peptides having d-amino acids as well as retro inverso peptides having d-amino acids.

TABLE 1
Illustrative Cell Permeating Peptides (CPPs)

| | SEQ |
|---|---|
| CPP Sequence | ID NO: |
| KFFKFFKFFK | 3 |
| CFFKDEL | 4 |
| KKLFKKILKYL | 5 |
| GRRRRRRRRRPPQ | 6 |
| KKLFKKILKYLKKLFKKILKYL | 7 |
| TRQARRNRRRRWRERQR | 8 |
| RRRRRRRRR | 9 |
| RRRRRRRRRRRR | 10 |
| KHKHKHKHKHKHKHKHKH | 11 |
| KKKKKKKKK | 12 |
| KKKKKKKKKKKKKKKKKK | 13 |
| RQIKIFFQNRRMKFKK | 14 |
| RKKRRRESRKKRRRES | 15 |
| GRKRKKRT | 16 |
| RKKRRQRRR | 17 |
| RRRQRRKKR | 18 |
| GLRKRLRKFRNKIKEK | 19 |
| KALKKLLAKWLAAAKALL | 20 |
| QLALQLALQALQAALQLA | 21 |
| LKTLATALTKLAKTLTTL | 22 |
| RAWMRWYSPTTRRYG | 23 |
| LLIILRRRIRKQAHAHSK | 24 |
| RQIRIWFQNRRMRWRR | 25 |
| MVTVLFRRLRIRRACGPPRVRV | 26 |
| RQIKIWFQNRRMKWKK | 27 |
| VRLPPPVRLPPPVRLPPP | 28 |
| LLLFLLKKRKKRKY | 29 |
| SYFILRRRRKRFPYFFTDVRVAA | 30 |
| RAGLQFPVGRVHRLLRK | 31 |
| IAARIKLRSRQHIKLRHL | 32 |
| SYDDLRRRRKRFPYFFTDVRVAA | 33 |
| KKALLALALHHLAHLALHLALALKKA | 34 |
| GLFKALLKLLKSLWKLLLKA | 35 |
| GWTLNSAGYLLGKINLKALAALAKKIL | 36 |
| GLFKALLKLLKSLWKLLLKAGLFKALLKLLKSLWKLLLKA | 37 |
| RQIKIWFPNRRMKWKK | 38 |
| QIKIWFQNRRMKWKK | 39 |
| KMDCRWRWKCCKK | 40 |
| MDCRWRWKCCKK | 41 |
| KCGCRWRWKCGCKK | 42 |
| CRWRWKCCKK | 43 |
| TKRRITPKDVIDVRSVTTEINT | 44 |
| AEKVDPVKLNLTLSAAAEALTGLGDK | 45 |
| TKRRITPKDVIDVRSVTTKINT | 46 |
| HHHHHHTKRRITPKDVIDVRSVTTEINT | 47 |
| GTKMIFVGIKKKEERADLIAYLKKA | 48 |
| KCFQWQRNMRKVRGPPVSCIKR | 49 |
| EEEAAGRKRKKRT | 50 |
| FLGKKFKKYFLQLLK | 51 |
| FLIFIRVICIVIAKLKANLMCKT | 52 |
| YIVLRRRRKRVNTKRS | 53 |
| KTVLLRKLLKLLVRKI | 54 |
| LLKKRKVVRLIKFLLK | 55 |
| KKICTRKPREMSAWAQ | 56 |
| GIGKFLHSAKKWGKAFVGQIMNC | 57 |
| LEDKFFC | 58 |

Any of the binding motifs may be indirectly or directly connected with any cell wall-permeating peptides (CPP) known in the art to form an antibacterial peptide. In various embodiments, the connection between the binding motif and the CPP includes a covalent bond, such as a peptide bond. In other embodiments, the connection between the binding motif and the CPP includes a covalent bond that does not include a peptide bond. In other embodiments, the connection between the binding motif and the CPP includes a linker of one or more atoms. Other direct or indirect linkages are possible. For example, suitable linkages are described in Lee et al., (Conjugation of Cell-Penetrating Peptides to Antimicrobial Peptides Enhances Antibacterial Activity. ACS Omega. 2019 Sep. 24; 4(13): 15694-15701) the disclosure of which is incorporated by reference in its entirety. In addition, the cell permeating peptide can be directly connected to the binding motif or may be separated by intervening amino acids. The cell permeating peptide can precede or follow the binding motif. Preferably, the cell permeating peptide links (directly or indirectly) to the N-terminus of the binding motif.

Any of the antibacterial peptides can be indirectly or directly connected with any cell wall-permeating peptides (CPP) known in the art to form an antibacterial peptide, including via any of the connections and linkages described above with respect to the connections between the binding motifs and CPPs. The cell permeating peptide can precede or follow the antibacterial peptides. Preferably, the cell permeating peptide links (directly or indirectly) to the N-terminus of the peptide.

As used herein, “peptide” is understood to be an amino acid chain that is notably shorter than a full-length protein. Accordingly, in various embodiments the antibacterial peptide can have a length of about 50 amino acids or fewer, about 45 amino acids or fewer, about 40 amino acids or fewer, about 35 amino acids or fewer, about 30 amino acids or fewer, about 25 amino acids or fewer, or about 20 amino acids or fewer. For example, the peptide can have a length of about 5 amino acids or greater, 6 amino acids or greater, 7 amino acids or greater, 8 amino acids or greater, 9 amino acids or greater, 10 amino acids or greater, about 11 amino acids or greater, about 12 amino acids or greater, about 13 amino acids or greater, about 14 amino acids or greater, about 15 amino acids or greater, about 16 amino acids or greater, about 17 amino acids or greater, about 18 amino acids or greater, or about 19 amino acids or greater. For example, the peptide can have a length from about 5 to about 50 amino acids, from about 5 to about 40 amino acids, from about 5 to about 30 amino acids, from about 5 to about 25 amino acids, or from about 5 to about 20 amino acids. In additional embodiments, the peptide can have a length of about 5, about 6, about 7, about 8, about 9, about 10, about 11, about 12, about 13, about 14, about 15, about 16, about 17, or about 18 amino acids.

As discussed above, the antibacterial peptide can inhibit the activity of a β-lactamase. Accordingly, the antibacterial peptide can bind to the β-lactamase. The β-lactamase can include an extended-spectrum β-lactamase. In addition, the binding of the antibacterial peptide to the β-lactamase can inhibit cleavage of a β-lactam by the β-lactamase.

In various embodiments, the β-lactamase can be expressed by a gram positive or a gram negative bacterium (bacteria).

When the β-lactamase is expressed by a gram positive bacterium, the gram positive bacterium can include Staphylococcus aureus, Streptococcus pneumoniae, Bacillus subtilis, Bacillus licheniformis, Bacillus cereus, Bacillus amyloliquefaciens, Bacillus velezensis, Bacillus thuringiensis, Bacillus mycoides, Streptomyces cellulosae, Streptomyces badius, Streptomyces cacaoi, Streptomyces fradiae (Streptomyces roseoflavus), Kitasatospora aureofaciens (Streptomyces aureofaciens), Streptomyces albus G, Streptomyces lavendulae, Nocardia, Amycolatopsis, Mycolicibacterium fortuitum (Mycobacterium fortuitum), Mycobacterium tuberculosis, or any combination thereof.

When the β-lactamase is expressed by a gram negative bacterium, the gram negative bacterium can include Escherichia coli, Neisseria gonorrhoeae, Acinetobacter baumanii, Moraxella catarrhalis, Shigella, Klebsiella, Enterobacter aerogenes, Enterobacter cloacae, Proteus, Mycolicibacterium fortuitum (Mycobacterium fortuitum), Mycobacterium tuberculosis, Aeromonas hydrophila, Pseudomonas aeruginosa, Stenotrophomonas maltophilia (Pseudomonas maltophilia), Rhodobacter capsulatus (Rhodopseudomonas capsulata), Haemophilus influenzae, Vibrio cholerae, Citrobacter, Yersinia, Serratia, Salmonella, Kluyvera, or any combination thereof.

In various embodiments, the β-lactamase is expressed by Escherichia coli or Staphylococcus aureus.

### Methods of Producing Antibacterial Peptides

Any of the peptides described herein can be prepared using standard methods in the art. For example, the peptides can be chemically synthesized via standard solid phase peptide synthesis described, for example, by Merrifield, R. B. (Solid Phase Peptide Synthesis I. The Synthesis of a Tetrapeptide. (1963) Journal of the American Chemical Society, 85, 2149-2154) the disclosure of which is incorporated by reference herein in its entirety. In various embodiments, the peptides provided herein may be modified to improve deliverability, stability, potency, or any other property important for drug delivery.

The peptides may be modified by either natural processes, such as posttranslational processing, or by chemical modification techniques that are well known in the art. Modifications can occur anywhere in a peptide, including the peptide backbone, the amino acid side-chains and the amino or carboxyl termini. The same type of modification may be present in the same or varying degrees at several sites in a peptide. Also, a peptide may contain many types of modifications.

Peptides may be branched, for example, as a result of ubiquitination, and they may be cyclic, with or without branching. Cyclic, branched, and branched cyclic polypeptides may result from posttranslation natural processes or may be made by synthetic methods.

Suitable chemical modifications include stapling, acetylation, acid addition, acylation, ADP-ribosylation, aldehyde addition, alkylamide addition, amidation, amination, biotinylation, carbamate addition, chloromethyl ketone addition, covalent attachment of a nucleotide or nucleotide derivative, cross-linking, cyclization, disulfide bond formation, demethylation, ester addition, formation of covalent cross-links, formation of cysteine-cysteine disulfide bonds, formation of pyroglutamate, formylation, gamma-carboxylation, glycosylation, GPI anchor formation, hydrazide addition, hydroxyamic acid addition, hydroxylation, iodination, lipid addition, methylation, myristoylation, oxidation, PEGylation, proteolytic processing, phosphorylation, prenylation, palmitoylation, addition of a purification tag, pyroglutamyl addition, racemization, selenoylation, sulfonamide addition, sulfation, transfer-RNA mediated addition of amino acids to proteins such as arginylation, ubiquitination, and urea addition. (see. e.g., Creighton et al. (1993) Proteins—Structure and Molecular Properties, 2nd Ed., T. E. Creighton, W. H. Freeman and Company. New York; Johnson, ed. (1983) Posttranslational Covalent Modification Of Proteins. Academic Press. New York; Seifter et al. (1990) Meth. Enzymol., 182: 626-646: Rattan et al. (1992) Ann. N.Y. Acad. Sci., 663: 48-62; and the like).

Using known methods of protein engineering and recombinant DNA technology, variants may be generated to improve or alter the characteristics of the peptides described herein. Such variants include deletions, insertions, inversions, repeats, duplications, extensions, and substitutions (e.g., conservative substitutions and/or substitutions with nonstandard amino acids) selected according to general rules well known in the art so as have little effect on activity.

### Compositions

Also provided herein is a composition comprising any antibacterial peptide described above and a phannaceutically appropriate excipient, a carrier and/or a drug delivery agent.

In various embodiments, the composition can include from about 0.01 to 500 μg/ml, from about 0.01 to 400 μg/ml, from about 0.01 to 300 μg/ml, from about 0.01 to 200 μg/ml, from about 0.01 to 190 μg/ml, from about 0.01 to 180 μg/ml, from about 0.01 to 170 g. ml, from about 0.01 to 160 μg/ml, from about 0.01 to 150 μg/ml, from about 0.01 to 140 μg/ml, or from about 0.01 to 130 μg/ml of the antibacterial peptide. For example, the composition can include from about 0.01 to 128 μg/ml of the antibacterial peptide.

Pharmaceutical compositions containing one or more of the antibacterial peptides described herein can be formulated in any conventional manner. Proper formulation is dependent in part upon the route of administration selected. Routes of administration include parenteral (e.g., intravenous, intra-arterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, oral, orthotopic, intratracheal, intralesional, percutaneous, endoscopical, transmucosal, sublingual and intestinal administration. Preferably, the composition is administered orally.

The compositions described herein can also include one or more pharmaceutically acceptable excipients and/or carriers. The pharmaceutically acceptable excipients and/or carriers for use in the compositions of the present disclosure can be selected based upon a number of factors including the particular compound used, and its concentration, stability and intended bioavailability; the subject, its age, size and general condition; and the route of administration. The peptides described herein may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces. These biologically active or inert agents can include, for example, enzyme inhibitors and absorption enhancers.

Some examples of materials which can serve as pharmaceutically acceptable carriers in the compositions described herein are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

The pharmaceutical compositions can be formulated for oral administration. The pharmaceutical compositions can be formulated as tablets, dispersible powders, pills, capsules, gel-caps, granules, solutions, suspensions, emulsions, syrups, elixirs, troches, lozenges, or any other dosage form that can be administered orally. The pharmaceutical compositions can include one or more pharmaceutically acceptable excipients. Suitable excipients for solid dosage forms include sugars, starches, and other conventional substances including lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, microcrystalline cellulose, colloidal silicon dioxide, croscarmellose sodium, talc, magnesium stearate, and stearic acid. Further, such solid dosage forms can be uncoated or can be coated to delay disintegration and absorption.

The pharmaceutical compositions can also be formulated for parenteral administration. e.g., formulated for injection via intravenous, intra-arterial subcutaneous, rectal, subcutaneous, intramuscular, intraorbital, intracapsular, intraspinal, intraperitoneal, or intrasternal routes. Dosage forms suitable for parenteral administration include solutions, suspensions, dispersions, emulsions or any other dosage form that can be administered parenterally.

Additional pharmaceutically acceptable excipients are identified, for example, in The Handbook of Pharmaceutical Excipients, (American Pharmaceutical Association, Washington, D.C., and The Pharmaceutical Society of Great Britain. London, England, 1968). Additional excipients can be included in the pharmaceutical compositions of the present disclosure for a variety of purposes. These excipients can impart properties winch enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, non-volatile silicones, adhesives, bulking agents, flavorings, sweeteners, adsorbents, binders, disintegrating agents, lubricants, coating agents, and antioxidants.

In addition, various drug delivery agents may be included in the compositions to facilitate delivery of the peptides to their target. These drug delivery agents include nanoparticles, microparticles, liposomes or others. The peptides can be covalently or non-covalently associated with the delivery vehicles via a linkage that may be suitably cleaved at the target.

In general, the compositions may be formulated to enhance the delivery of the peptides according to standard procedures in the art. Procedures for delivering peptides are described, for example in Bruno et al., (Basics and recent advances in peptide and protein drug delivery. Ther Deliv. 2013; 4(11): 1443-1467) and in Jitendra et al., (Noninvasive Routes of Proteins and Peptides Drug Delivery, Indian J Pharm Sci. 2011:73(4):367-75). The disclosures of Bruno et al. and Jitendra et al. are incorporated herein by reference in their entirety.

In addition, the composition can further include an antibiotic having a β-lactam ring. The antibiotic can include a penicillin, a carbapenem or a panem. For example, the antibiotic can include Benzylpenicillin, Benzathine benzylpenicillin, Procaine benzylpenicillin, Phenoxymethylpenicillin, Propicillin, Pheneticillin, Azidocillin, Clometocillin, Penamecillin, Cloxacillin, Oxacillin, Nafcillin, Methicillin, Amoxicillin, Ampicillin, Epicillin, Ticarcillin, Carbenicillin, Carindacillin, Temocillin, Piperacillin, Azlocillin, Mezlocillin, Mecillinam, Sulbenicillin, Faropenem, Ritipenem, Ertapenem Antipseudomonal, Biapenem, Panipenem, Cefazolin, Cefalexin, Cefadroxil, Cefapirin, Cefazedone, Cefazaflur, Cefradine, Cefroxadine, Ceftezole, Cefaloglycin, Cefacetrile, Cefalonium, Cefaloridine, Cefalotin, Cefatrizine, Cefixime, Ceftriaxone, Cefotaxime, Cefdinir, Cefcapene, Cefdaloxine, Ceftizoxime, Cefmenoxime, Cefpiramide, Cefpodoxime, Ceftibuten, Cefditoren, Cefotiam, Cefetamet, Cefodizime, Cefpimizole, Cefsulodin, Cefteram, Ceftiolene, Oxacephem, Cefepime, Cefozopran, Cefpirome, Cefquinome, Ceftaroline fosamil, Ceftolozane, Ceftobiprole, Ceftiofur, Cefquinome, Cefovecin, Aztreonam, Tigemonam, Carumonam, Nocardicin A, Doribax, Invanz, Merrem IV, Imipenem/Cilastatin, Meropenem/Vaborbactam, Imipenem/Cilastatin/Relebactam, Primaxin, Recarbrio, Vabomere, Imipenem, Panipenem/betamipron, Tebipenem, Ertapenem, Doripenem, Meropenem, Faropenem, Ritipenem, a prodrug thereof, or any combination thereof. Preferably, the antibiotic includes a penicillin or a carbapenem. For example, the antibiotic can include amoxicillin or a carbapenem.

Methods of Decreasing Bacterial Titer and/or Treating Bacterial Infection

Also provided are methods of reducing a bacterial titer. Also provided are medicaments comprising the peptides or compositions described above in the use of reducing a bacterial titer.

The method of reducing a bacterial titer includes applying any of the antibacterial peptides or compositions having the antibacterial peptides as described above to the bacteria. In various embodiments, the bacteria are located within a subject (i.e., an animal, plant, or other organism). Accordingly, the method may further include administering the antibacterial peptide or composition comprising the antibacterial peptide to the subject. In various embodiments, the peptides include a CPP to enhance damage to the bacterial cell membrane.

In various embodiments, the method may further include applying an antibiotic to the bacteria, or administering an antibiotic to the subject. The antibiotic preferably includes a β-lactam ring. Adding β-lactam with the peptide can significantly enhance bactericidal effect compared to peptide alone. For example, the antibiotic can include any antibiotic described herein above. In various embodiments, the method may include applying or administering another β-lactamase inhibitor drug to the bacteria or subject, either in combination with an antibiotic or without an antibiotic. The antibiotic and additional β-lactamase inhibitor drug can independently be applied or administered in the same composition as the antibacterial peptides or in one or more separate compositions, which may be applied or administered simultaneously or sequentially.

The target bacteria may show resistance to the antibiotic. That is, it may show less sensitivity to the antibiotic's effect on its growth rate, replication rate, virulence, or other some other measure known in the art as compared to a bacterium that has not developed resistance to the antibiotic. One way to measure the bacteria's sensitivity can be to measure the minimum inhibitor concentration (MIC) of the antibiotic against the bacteria according to Clinical and Laboratory Standards Institute protocols. For example, one protocol is described in the following document: CLSI. Methods for Dilution Antimicrobial Susceptibility Tests for Bacteria That Grown Aerobically; Approved Standard—Tenth Edition. CLSI document M07-A10. Wayne PA: Clinical and Laboratory Standards Institute, 2015, incorporated herein by reference in its entirety. The antibacterial peptide may increase sensitivity (decrease resistance) to the antibiotic. For example, the antibacterial peptide can decrease the minimum inhibitory concentration (MIC) of the antibiotic against the bacteria as compared to the MIC of the antibiotic without the antibacterial peptide.

The bacteria can express a β-lactamase and the antibacterial peptide can inhibit the ability of the β-lactamase to cleave a β-lactam ring. This can be measured using a beta lactamase inhibition assay like a commercially available beta lactamase inhibitor screening kit. These kits test the ability of a beta lactamase to hydrolyze a chromogenic substrate, which results in the generation of a colored product. The amount of color produced is directly proportional to the amount of beta-lactamase activity. In the presence of beta lactamase inhibitors, such as clavulanic acid or the antibacterial peptides described herein, the rate of substrate hydrolysis will decrease resulting in a decrease in the production of colored analyte. The non-peptide β-lactamase inhibitor can include clavulanate, clavulanic acid, sulbactam, taobactam, avibactam, relebacam, RG6080, or RPX7009.

The bacteria can include any gram negative or gram positive bacteria described herein above. For example, the bacteria can include Escherichia coli or Staphylococcus aureus.

In various embodiments, the methods can further include treating a bacterial infection in a subject in need thereof. The method of treating a bacterial infection can include administering an effective amount of the antibacterial peptide or compositions comprising the antibacterial peptide to the subject, as described above. The method may further include administering an antibiotic, another β-lactamase inhibitor drugs, or combinations thereof to the subject, as described above. The subject can be an animal or a plant. In some embodiments, the subject is an animal (i.e., a human).

The bacterial infection can be caused by any gram negative or gram positive bacteria described above. For example, the bacteria can include Escherichia coli, Acinetobacter baumannii, Neisseria gonorrhoeae, Moraxella catarrhalis, Shigella, Klebsiella, Enterobacter aerogenes, Enterobacter cloacae, Proteus, Mycolicibacterium fortuitum (Mycobacterium fortuitum), Myobacterium tuberculosis, Aeromonas hydrophila, Pseudomonas aeruginosa, Stenotrophomonas maltophilia (Pseudomonas maltophilia), Rhodobacter capsulatus (Rhodopseudomonas capsulata), Haemophilus influenzae, Vibrio cholerae, Citrobacter, Yersinia, Serratia, Salmonella, Kluyvera, Staphylococcus aureus, Streptococcus pneumoniae, Bacillus subtilis, Bacillus licheniformis, Bacillus cereus, Bacillus amyloliquefaciens (Bacillus velezensis), Bacillus thuringiensis, Bacillus mycoides, Streptomyces cellulosae, Streptomyces badius, Streptomyces cacaoi, Streptomyces fradiae (Streptomyces roseoflavus), Kitasatospora aureofaciens (Streptomyces aureofaciens), Streptomyces albus G, Streptomyces lavendulae, Nocardia, Amycolatopsis, Mycolicibacterium fortuitum (Mycobacterium fortuitum), Mycobacterium tuberculosis, or any combination thereof.

When the infection occurs in an animal system (e.g., when the subject is an animal), it can occur in any organ system, including but not limited to, the digestive system, the cardiovascular system, the respiratory system, or the reproductive system.

The effective amount of the antibacterial peptide can depend on whether the peptide is administered in vivo (i.e., in a subject to treat a bacterial infection) or in vitro (i.e., to reduce bacterial titer in a dish). In vitro, an effective amount of the antibacterial peptide can include from about 0.01 to 500 μg/ml, from about 0.01 to 400 μg/ml, from about 0.01 to 300 μg/ml, from about 0.01 to 200 μg/ml, from about 0.01 to 190 μg/ml, from about 0.01 to ISO μg/ml, from about 0.01 to 170 μg/ml, from about 0.01 to 160 μg/ml, from about 0.01 to 150 μg mi, from about 0.01 to 140 μg/ml, or from about 0.01 to 130 μg/ml of the antibacterial peptide. For example, the effective amount can include from about 0.01 to 128 μg/ml of the antibacterial peptide. In vivo, the effective amount of the antibacterial peptide can include from about 0.01 to 1000 mg/kg, from about 0.01 to 900 mg/kg, from about 0.01 to 800 mg/kg, from about 0.01 to 700 mg/kg, from about 0.01 to 600 mg/kg, or from about 0.01 to 500 mg/kg. For example, the effective amount of the antibacterial peptide can include from about 0.01 to 500 mg/kg.

The method can further include administering an antibiotic to the subject. The antibiotic can include any antibiotic described herein above (i.e., include a ft-lactam ring). The method can cover any method or sequence of administration of the antibiotic and the antibacterial peptide. For example, the antibiotic and antibacterial peptide can be administered separately or together. The antibiotic can be administered before the peptide or vice-versa.

### EXAMPLES

The following non-limiting examples are provided to further illustrate the present invention.

### Example 1: D-Amino Add Substitutions in T61-25 Candidate Improve Serum Stability and Maintained its TEM-1 Inhibition

D-amino acids were introduced into the peptide to determine the effect on the stability of T61-25. Computational modeling suggested that four residues (i.e., T2, A5, Q6, and A9) are on the opposite side of the TEM-1-binding interface and thus can be subjected to D-amino acid substitution. Four peptides were synthesized, each with one of these positions substituted by their corresponding D-amino acids idT2, dA5, dQ6, and dA9, respectively). A fully modified T61-25 peptide with all D-amino acids in the forward (D-T61-25) and retro inverso (RI) sequences (RI-D-T61-25) were also synthesized. The β-lactamase colorimetry assay showed that dT2. D-T61-25, and RI-D-T61-25 all maintained comparable levels of TEM-1 inhibition as T61-25 (Table 1). Since the Ki of RI-D-T61-25 was nearly as good as T61-25, a cell-penetrating peptide (CPP) BP100 was conjugated to T61-25 and synthesized the L-peptide and RI-D peptide (BP100-T61-25 and RI-D-BP100-T61-25, respectively). Table 2 summarizes results showing that the serum stability of the RI-D-BP100-T61-25 was better than the L-peptide BP100-T61-25, indicating that the RI-D peptide can be a good drug candidate that will be more stable in vivo (FIG. 1).

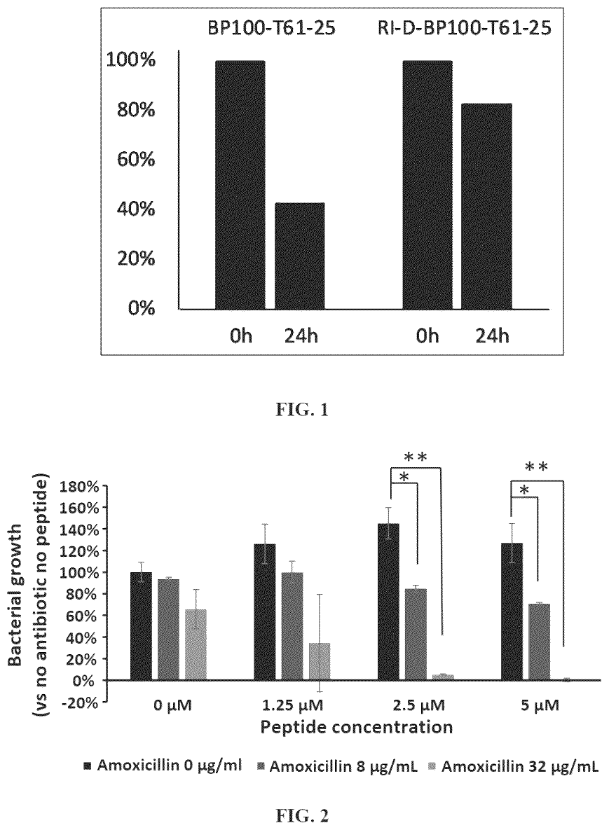

The potency of RI-D-BP100-T61-25 was tested with the E. coli strain ATCC35218, which is resistant to amoxicillin by expressing TEM-1 β-lactamase (FIG. 2)(CLSI. Methods for Dilution Antimicrobial Susceptibility Tests for Bacteria That Grow Aerobically; Approved Standard—Tenth Edition. Wayne. PA 19087 USA: Clinical and Laboratory Standards Institute; 2015). The peptide demonstrated significant inhibition of ATCC35218 growth in combination with 8 μg/mL amoxicillin (FIG. 2). Complete inhibition of bacterial growth was achieved at 2.5 μM peptide with 32 μg/mL amoxicillin (FIG. 2), which was chosen since it is non-resistant Amoxicillin-Clavulanate breakpoint for E. coli (FDA. Breakpoints. U.S. Food and Drug Administration); CLSL M100-S24: Performance Standards for Antimicrobial Susceptibility Testing; Twenty-Fourth Informational Supplement. Wayne, PA 19087 USA: Clinical and Laboratory Standards Institute; 2014), the concentration at which a species of bacteria is susceptible or resistance to an antibiotic. These data indicated that D-amino acid substitution effectively improved peptide serum stability and achieved desirable biological activity against drug resistant gram-negative bacteria.

TABLE 2
Ki of D-amino acid substituted peptides in
inhibiting TEM-1.

| | Sequence | |
|---|---|---|
| Inhibitor | (SEQ ID NO:) | TEM-1 Ki (μM) |
| RI-D-T61-25 | all-d(GTAAAQALYTK) | 0.88 ± 0.04 |
| | (SEQ ID NO: 1) | |
| T61-25 | KTYLAQAAATG | 0.39 ± 0.18 |
| | (SEQ ID NO: 59) | |
| D-T2 | K-dT-YLAQAAATG | 5.63 ± 0.96 |
| | (SEQ ID NO: 60) | |
| D-T61-25 | all-d(KTYLAQAAATG) | 2.18 ± 0.55 |
| | (SEQ ID NO: 61) | |

### Example 2: RI-D-BP100-T61-25 Inhibited PBP2a Activity and Overcome β-Lactam Resistance in MRSA

Methicillin-resistant Staphylococcus aureus (MRSA) contains the mecA gene in a mobile genetic element found in all MRSA strains, which encodes penicillin-binding protein 2a (PBP2a). PBPs are membrane-bound enzymes that catalyze the transpeptidation reaction that is necessary for cross-linkage of peptidoglycan chains for cell wall formation and targeted by β-lactam. Because of the low affinity of PBP2a for all β-lactam antibiotics, it can substitute for other PBPs under high concentrations of f-lactam antibiotics. As a result. MRSA strains are highly resistant to β-lactam antibiotics.

RI-D-BP100-T61-25 was also examined for its effect on PBP2a activity due to the similarity of the β-lactam binding pockets between PBP2a and TEM-1. The PBP2a colorimetry assay showed that T61-25 and RI-D-BP100-T61-25 were capable of inhibiting PBP2a activity at μM range (Table 3).

TABLE 3
Ki of D-amino acid substituted peptides in
inhibiting PBP2a.

| | Sequence | PBP2a Kᵢ |
|---|---|---|
| Inhibitor | (SEQ ID NO:) | (μM) |
| RI-D-BP100- | all- | 3.7 ± 0.7 |
| T61-25 | d(GTAAAQALYTKLYKLIKKFLKK) | |
| | (SEQ ID NO: 2) | |
| T61-25 | KTYLAQAAATG | 9.7 ± 7.9 |
| | (SEQ ID NO: 62) | |

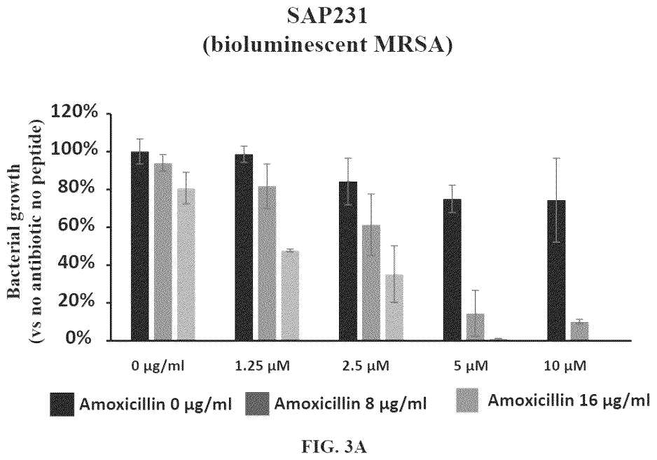

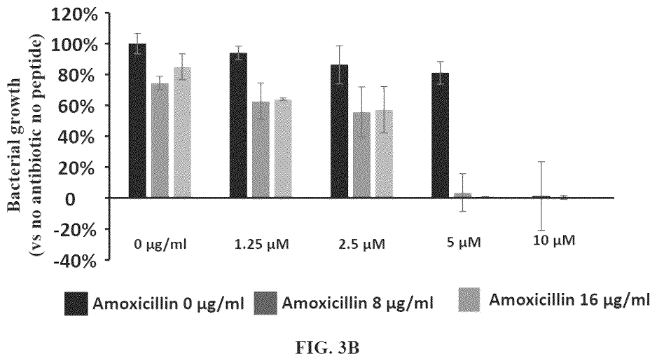

The peptide's potency at enhancing β-lactam antibiotics' killing of SAP231 strain, which is a bioluminescent strain of the MRSA strain NRS384 (Plaut R D, Mocca C P, Prabhakara R, Merkel T J, Stibitz S. Stably luminescent Staphylococcus aureus clinical strains for use in bioluminescent imaging. PloS one. 2013; 8(3):e59232), was tested. RI-D-BP100-T61-25 at concentration as low as 1.25 μM was able to significantly enhance the amoxicillin's inhibition of MRSA growth. Complete inhibition of bacterial growth was achieved at 5 μM peptide with 16 μg/mL amoxicillin. RI-D-BP100-T61-25 was combined with amoxicillin and tested against both SAP231 (FIG. 3A) and NRS384 strain (FIG. 3B).

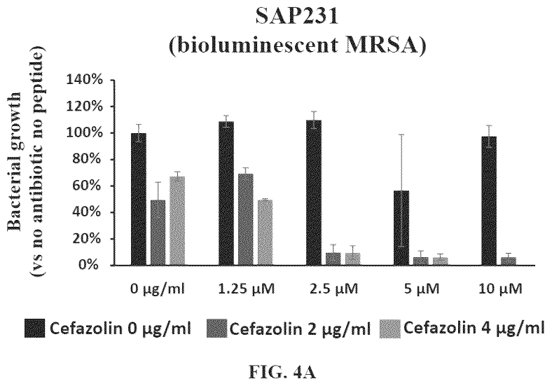

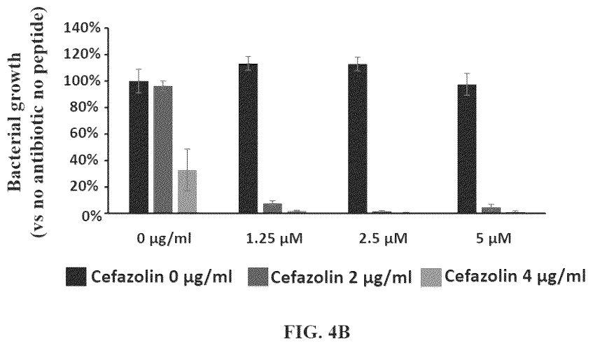

Additionally, the peptide's potency at enhancing cefazolin killing of MRSA was also tested. Cefazolin is a cephalosporin, which are improved β-lactams developed to overcome some of the early penicillin resistance. Cefazolin is a first line therapy for the treatment of Methicillin-susceptible Staphylococcus aureus (MSSA) infections. Although cefazolin could partially inhibited SAP231 growth at the test concentrations (0 μg/ml peptide), RI-D-BP100-T61-25 at concentration ≥2.5 μM was able to mostly kill MRSA with cefazolin (2 and 4 μg/ml). RI-D-BP100-T61-25 was also combined with cefazolin and tested against both SAP231 (FIG. 4A) and NRS384 strain (FIG. 4B).

These results indicate that the candidate peptide can achieve desirable biological activity against MRSA.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

1. An antibacterial peptide comprising a binding motif that comprises an amino acid sequence selected from the group consisting of:
dGdTdAdAdAdQdAdLdYdTdK (SEQ ID NO:1),
dGdTdAdAdAdQdAdLdYdTdKdLdYdKdLdIdKdKdFdLdKdK (SEQ ID NO:2), and
combinations thereof.

2. The antibacterial peptide of claim 1, wherein the antibacterial peptide further comprises a cell-wall permeating peptide.

3. The antibacterial peptide of claim 2, wherein the cell wall permeating peptide comprises any one of SEQ ID NOs: 3-58.

4. The antibacterial peptide of claim 3, wherein the cell wall permeating peptide is selected from the group consisting of KFFKFFKFFK (SEQ ID NO: 3), CFFKDEL (SEQ ID NO:4) KKLFKKILKYL (SEQ ID NO:5), and GRRRRRRRRRPPQ (SEQ ID NO:6).

5. The antibacterial peptide of claim 1, wherein the antibacterial peptide:
(a) further comprises a chemical modification selected from the group consisting of stapling, acetylation, acid addition, acylation, ADP-ribosylation, aldehyde addition, alkylamide addition, amidation, amination, biotinylation, carbamate addition, chloromethyl ketone addition, covalent attachment of a nucleotide or nucleotide derivative, cross-linking, cyclization, disulfide bond formation, demethylation, ester addition, formation of covalent cross-links, formation of cysteine-cysteine disulfide bonds, formation of pyroglutamate, formylation, gamma-carboxylation, glycosylation, GPI anchor formation, hydrazide addition, hydroxyamic acid addition, hydroxylation, iodination, lipid addition, methylation, myristoylation, oxidation, PEGylation, proteolytic processing, phosphorylation, prenylation, palmitoylation, addition of a purification tag, pyroglutamyl addition, racemization, selenoylation, sulfonamide addition, sulfation, transfer-RNA mediated addition of amino acids to proteins, and combinations thereof,
(b) is a variant comprising at least one insertion, deletion or substitution;
(c) is chemically synthesized with D-amino acids, β2-amino acids, β3-amino acids, homo amino acids, gamma amino acids, peptoids, N-methyl amino acids, and/or other non-natural amino acid mimics and derivatives;
(d) is cyclized or branched; or
(e) any combination thereof.

6. The antibacterial peptide of claim 1, wherein the peptide has a length from about 5 to about 50 amino acids or fewer.

7. (canceled)

8. The antibacterial peptide of claim 1, wherein the composition comprises from about 0.01 μg/ml to about 128 μg/ml of the peptide.

9. The antibacterial peptide of claim 1, further comprising an antibiotic comprising a β-lactam ring.

10.-12. (canceled)

13. A method of reducing a titer of bacteria, the method comprising applying an effective amount of the antibacterial peptide of claim 1 to the bacteria.

14. The method of claim 13, wherein the bacteria are present in a subject and the method comprises administering the antibacterial peptide to the subject.

15. The method of claim 13, wherein the bacteria have reduced sensitivity to an antibiotic, and wherein the antibacterial peptide increases the sensitivity of the bacteria to the antibiotic.

16. The method of claim 15, wherein the antibiotic comprises a β-lactam ring.

17. The method of claim 13, wherein the bacteria comprise a β-lactamase expressing bacterium.

18. The method of claim 17, wherein the antibacterial peptide inhibits the activity of the β-lactamase.

19. The method of claim 18, wherein the antibacterial peptide inhibits the activity of the β-lactamase more than a non-peptide β-lactamase inhibitor.

20. The method of claim 19, wherein the non-peptide β-lactamase inhibitor comprises clavulanate.

21. The method of claim 13, wherein the bacteria is selected from a gram negative bacterium, a gram positive bacterium, and combinations thereof.

22. The method of claim 14, further comprising treating a bacterial infection in the subject.

23. The method of claim 22, wherein the bacterial infection is caused by bacteria selected from the group consisting of Escherichia coli, Acinetobacter baumannii, Neisseria gonorrhoeae, Moraxella catarrhalis, Shigella, Klebsiella, Enterobacter cloacae, Enterobacter aerogenes Proteus, Mycolicibacterium fortuitum (Mycobacterium fortuitum), Mycobacterium tuberculosis, Aeromonas hydrophila, Pseudomonas aeruginosa, Stenotrophomonas maltophilia (Pseudomonas maltophilia), Rhodobacter capsulatus (Rhodopseudomonas capsulata), Haemophilus influenzae, Vibrio cholerae, Citrobacter, Yersinia, Serratia, Salmonella, Kluyvera, Staphylococcus aureus, Streptococcus pneumoniae, Bacillus subtilis, Bacillus lichenmformis, Bacillus cereus, Bacillus amyloliquefaciens (Bacillus velezensis), Bacillus thuringiensis, Bacillus mycoides, Streptomyces cellulosae, Streptomyces badius, Streptomyces cacaoi, Streptomyces fradiae (Streptomyces roseoflavus), Kitasatospora aureofaciens (Streptomyces aureofaciens), Streptomyces albus G, Streptomyces lavendulae, Nocardia, Amycolatopsis, Mycolicibacterium fortuitum (Mycobacterium fortuitum), Mycobacterium tuberculosis, and combinations thereof.

24. The method of claim 13, wherein the effective amount of the antibacterial peptide comprises from about 0.01 μg/ml to 128 μg/ml.

25.-28. (canceled)


SEQUENCE LISTING (62 sequences)

SEQ ID NO: 1 - protein, 11 aa, synthetic construct
  SITE 1: note = D-glycine
  SITE 2: note = D-threonine
  SITE 3: note = D-alanine
  SITE 4: note = D-alanine
  SITE 5: note = D-alanine
  SITE 6: note = D-glutamine
  SITE 7: note = D-alanine
  SITE 8: note = D-leucine
  SITE 9: note = D-tyrosine
  SITE 10: note = D-threonine
  SITE 11: note = D-lysine
GTAAAQALYTK

SEQ ID NO: 2 - protein, 22 aa, synthetic construct
  SITE 1: note = D-glycine
  SITE 2: note = D-threonine
  SITE 3: note = D-alanine
  SITE 4: note = D-alanine
  SITE 5: note = D-alanine
  SITE 6: note = D-glutamine
  SITE 7: note = D-alanine
  SITE 8: note = D-leucine
  SITE 9: note = D-tyrosine
  SITE 10: note = D-threonine
  SITE 11: note = D-lysine
  SITE 12: note = D-leucine
  SITE 13: note = D-tyrosine
  SITE 14: note = D-lysine
  SITE 15: note = D-leucine
  SITE 16: note = D-isoleucine
  SITE 17: note = D-lysine
  SITE 18: note = D-lysine
  SITE 19: note = D-phenylalanine
  SITE 20: note = D-leucine
  SITE 21: note = D-lysine
  SITE 22: note = D-lysine
GTAAAQALYTKLYKLIKKFLKK

SEQ ID NO: 3 - protein, 10 aa, synthetic construct
KFFKFFKFFK

SEQ ID NO: 4 - protein, 7 aa, synthetic construct
CFFKDEL

SEQ ID NO: 5 - protein, 11 aa, synthetic construct
KKLFKKILKYL

SEQ ID NO: 6 - protein, 13 aa, synthetic construct
GRRRRRRRRRPPQ

SEQ ID NO: 7 - protein, 22 aa, synthetic construct
KKLFKKILKYLKKLFKKILKYL

SEQ ID NO: 8 - protein, 17 aa, synthetic construct
TRQARRNRRRRWRERQR

SEQ ID NO: 9 - protein, 9 aa, synthetic construct
RRRRRRRRR

SEQ ID NO: 10 - protein, 12 aa, synthetic construct
RRRRRRRRRRRR

SEQ ID NO: 11 - protein, 18 aa, synthetic construct
KHKHKHKHKHKHKHKHKH

SEQ ID NO: 12 - protein, 9 aa, synthetic construct
KKKKKKKKK

SEQ ID NO: 13 - protein, 18 aa, synthetic construct
KKKKKKKKKKKKKKKKKK

SEQ ID NO: 14 - protein, 16 aa, synthetic construct
RQIKIFFQNRRMKFKK

SEQ ID NO: 15 - protein, 16 aa, synthetic construct
RKKRRRESRKKRRRES

SEQ ID NO: 16 - protein, 8 aa, synthetic construct
GRKRKKRT

SEQ ID NO: 17 - protein, 9 aa, synthetic construct
RKKRRQRRR

SEQ ID NO: 18 - protein, 9 aa, synthetic construct
RRRQRRKKR

SEQ ID NO: 19 - protein, 16 aa, synthetic construct
GLRKRLRKFRNKIKEK

SEQ ID NO: 20 - protein, 18 aa, synthetic construct
KALKKLLAKWLAAAKALL

SEQ ID NO: 21 - protein, 18 aa, synthetic construct
QLALQLALQALQAALQLA

SEQ ID NO: 22 - protein, 18 aa, synthetic construct
LKTLATALTKLAKTLTTL

SEQ ID NO: 23 - protein, 15 aa, synthetic construct
RAWMRWYSPTTRRYG

SEQ ID NO: 24 - protein, 18 aa, synthetic construct
LLIILRRRIRKQAHAHSK

SEQ ID NO: 25 - protein, 16 aa, synthetic construct
RQIRIWFQNRRMRWRR

SEQ ID NO: 26 - protein, 22 aa, synthetic construct
MVTVLFRRLRIRRACGPPRVRV

SEQ ID NO: 27 - protein, 16 aa, synthetic construct
RQIKIWFQNRRMKWKK

SEQ ID NO: 28 - protein, 18 aa, synthetic construct
VRLPPPVRLPPPVRLPPP

SEQ ID NO: 29 - protein, 14 aa, synthetic construct
LLLFLLKKRKKRKY

SEQ ID NO: 30 - protein, 23 aa, synthetic construct
SYFILRRRRKRFPYFFTDVRVAA

SEQ ID NO: 31 - protein, 17 aa, synthetic construct
RAGLQFPVGRVHRLLRK

SEQ ID NO: 32 - protein, 18 aa, synthetic construct
IAARIKLRSRQHIKLRHL

SEQ ID NO: 33 - protein, 23 aa, synthetic construct
SYDDLRRRRKRFPYFFTDVRVAA

SEQ ID NO: 34 - protein, 26 aa, synthetic construct
KKALLALALHHLAHLALHLALALKKA

SEQ ID NO: 35 - protein, 20 aa, synthetic construct
GLFKALLKLLKSLWKLLLKA

SEQ ID NO: 36 - protein, 27 aa, synthetic construct
GWTLNSAGYLLGKINLKALAALAKKIL

SEQ ID NO: 37 - protein, 40 aa, synthetic construct
GLFKALLKLLKSLWKLLLKAGLFKALLKLLKSLWKLLLKA

SEQ ID NO: 38 - protein, 16 aa, synthetic construct
RQIKIWFPNRRMKWKK

SEQ ID NO: 39 - protein, 15 aa, synthetic construct
QIKIWFQNRRMKWKK

SEQ ID NO: 40 - protein, 13 aa, synthetic construct
KMDCRWRWKCCKK

SEQ ID NO: 41 - protein, 12 aa, synthetic construct
MDCRWRWKCCKK

SEQ ID NO: 42 - protein, 14 aa, synthetic construct
KCGCRWRWKCGCKK

SEQ ID NO: 43 - protein, 10 aa, synthetic construct
CRWRWKCCKK

SEQ ID NO: 44 - protein, 22 aa, synthetic construct
TKRRITPKDVIDVRSVTTEINT

SEQ ID NO: 45 - protein, 26 aa, synthetic construct
AEKVDPVKLNLTLSAAAEALTGLGDK

SEQ ID NO: 46 - protein, 22 aa, synthetic construct
TKRRITPKDVIDVRSVTTKINT

SEQ ID NO: 47 - protein, 28 aa, synthetic construct
HHHHHHTKRRITPKDVIDVRSVTTEINT

SEQ ID NO: 48 - protein, 25 aa, synthetic construct
GTKMIFVGIKKKEERADLIAYLKKA

SEQ ID NO: 49 - protein, 22 aa, synthetic construct
KCFQWQRNMRKVRGPPVSCIKR

SEQ ID NO: 50 - protein, 13 aa, synthetic construct
EEEAAGRKRKKRT

SEQ ID NO: 51 - protein, 15 aa, synthetic construct
FLGKKFKKYFLQLLK

SEQ ID NO: 52 - protein, 23 aa, synthetic construct
FLIFIRVICIVIAKLKANLMCKT

SEQ ID NO: 53 - protein, 16 aa, synthetic construct
YIVLRRRRKRVNTKRS

SEQ ID NO: 54 - protein, 16 aa, synthetic construct
KTVLLRKLLKLLVRKI

SEQ ID NO: 55 - protein, 16 aa, synthetic construct
LLKKRKVVRLIKFLLK

SEQ ID NO: 56 - protein, 16 aa, synthetic construct
KKICTRKPRFMSAWAQ

SEQ ID NO: 57 - protein, 23 aa, synthetic construct
GIGKFLHSAKKWGKAFVGQIMNC

SEQ ID NO: 58 - protein, 7 aa, synthetic construct
LEDKFFC

SEQ ID NO: 59 - protein, 11 aa, synthetic construct
KTYLAQAAATG

SEQ ID NO: 60 - protein, 11 aa, synthetic construct
  SITE 2: note = D-threonine
KTYLAQAAATG

SEQ ID NO: 61 - protein, 11 aa, synthetic construct
  SITE 1: note = D-lysine
  SITE 2: note = D-threonine
  SITE 3: note = D-tyrosine
  SITE 4: note = D-leucine
  SITE 5: note = D-alanine
  SITE 6: note = D-glutamine
  SITE 7: note = D-alanine
  SITE 8: note = D-alanine
  SITE 9: note = D-alanine
  SITE 10: note = D-threonine
  SITE 11: note = D-glycine
KTYLAQAAATG

SEQ ID NO: 62 - protein, 11 aa, synthetic construct
KTYLAQAAATG